(19) United States
(12) Patent Application Publication
(10) Pub. No.: US 2018/0110997 A1
(43) Pub. Date: Apr. 26, 2018

(54) Magnetometers

(71) Applicant: Brandon Nedelman, (US)

(72) Inventor: Brandon Nedelman, Oceanside, CA (US)

(21) Appl. No.: 15/838,382

(22) Filed: Dec. 12, 2017

(51) Int. Cl. A61N 2/00; A61N 2/02

(52) U.S. Cl. CPC A61N 2/004; G01R 33/0041; A61N 2/02

(57) ABSTRACT

Therapy, involving a long ranged magnetometer including auditory speech hallucinations and medical mood experiences.

### BACKGROUND ART

Far-field, wireless means of achieving brainwave readings have been achieved by technology such as SQUID or magnetometer. A magnetometer can be, in advanced applications, able to take brainwave measurements in hertz from an animal on the earth, at a long distance away located in orbital, outer space. Such technology examples termed are EEG, MEG, MRI, FMRI, etc. The atomic magnetometer functions as a long distance, calibrated, and beamformed (directionally propelled) means of taking neurological biofeedback and brain acoustic readings. The application of a satelliteatomic-magnetometer both eliminates the need for implantable or scalp mounted medical electrodes, and provides means for advanced scientific applications.

A magnetometer is a type of transducer. The satellite is assembled with a very large and powerful electromagnetic induction coil. On transducers such as microphones (input) or speakers (output), this can commonly be referred to as a voice coil. The function of the coil is to facilitate the input or output of the transduction device by causing the conversion of applied electromagnetic signals to be outputted from the device as sound or hertz signals. A microphone and speaker can be interchangeable, by simple reversing the polarity of the circuit applied to the transducer. If one is to reverse the polarity of the circuit of a microphone, it essentially becomes a speaker.

### APPLICATION OF THIS INVENTION

Advanced brainwave and psychoacoustic readings are taken from magnetometer applications by projecting a sensing or reading magnetic probe field at the brain of the animal. This is a hertz based wave that is similar to sound spectral wave types. Said is commonly used in functional brainwave and neurofeedback reading applications.

This invention reverses the polarity of the circuit of the magnetometer to cause an output signal or wave osscilation to be induced rather than an input or

sensing/reading. Such allows binaural or induction signals to be induced input to the animal brain, rather than a sensory reading or measurement.

The said induction output is a hertz based wave compatible with animal neuro osscilations. It is similar/a type of sound wave, however the properties of the osscilating wave (sine, square, saw, etc) are tuned in waveform properties such as hertz, tesla, etc to not be directly audible via the ears of the animal in its wave range. Rather, the osscilation is so tuned to be highly magnetic and not within applicable measurements of standard type sound waves that are detectable via mammal ear vibration sensing. It is essentially a reversed mri, fmri, eeg, meg, wavetype. Since the polarity of the magnetometer is reversed from the sensing/detecting type, the magnetic field or hertz osscilation is rather projected, from a distance, to the receiving animal. Such outputted osscilation is detectable by the receiving animals brain, although not through the ears.

Hearing is mentioned and applied as in many patents and literatures from sources such as regents of the university of California, and government sponsored research programs such as DARPA silent talk that utilize algorithms and means of using brainwave reading signals and converting the detected osscilations into equivalent sounds and translations of imagined speech. There are many interconnected brain components that are involved of the processing of spoken, imagined, and heard speech processing. Such examples of the large number of interconnected components include the audio cortex and nerves junctioned to the throat and mouth muscles. It is theorized that if a waveform is properly configured via this application, there is no specific need to technically probe a single component of the brain. Simply a brain within the range of the generated osscilating magnetic field will detect the induction as a sound or sensation that is more or less audibly perceivable, regardless of whether or not actually hearing involving standard reception via the ears is involved.

Algorithms exist for the decoding of imagined thoughts, speech, and sound that aren't spoken or heard by a person, into an output means such as screen displayed text translations, automated sound samples from a library, or even custom scenario sound generations, based on the thought processing and imagination of the brain. Examples of this application include implantable hearing aids, brain interfaces, thought speech decoding, etc.

Regarding the generation of sound, this invention calls for a means of applying an audio electric signal, such as generated by the input into a microphone, to be assembled to via computer/hardware/software means, cause the satellite atomic magento-inducer to use a reversed version of the decoding/generating technology to, output the inputted sound wave as an osscilation magnetic field. Rather than reading and decoding a brainwave neural activity into an audio equivalent, this invention will provide the reversed application. One can cause a sound signal to interact with the brain so to provide an entry or perception, rather than a measurement or reading. Enhancing medically cochlear implantable hearing aids, this application can be used remotely, discretely, and from outer space. Space and military applications of this invention are provided for.

A combination assembly of 1 means of magneto-sensing of speech/thought neuroosscilations, and 1 means of inducing or transmitting neuro-osscilations, can be applied. Therefore, two-way communication is possible providing for satellite governed wireless telepathy via the said switched polarity application of a magenetocoil-transducer.

Alpha Wave Readings/Induction:

Alerted brain states of awakening are predominated by alpha brainwaves. The alerted state, which may be facilitated by the consumption of stimulant bioactive agents such as amphetamines, cause a dominating said state. It is known that alpha level brainwaves are increased, in terms of readability by a magneto reading application.

The induction or output of alpha level osscilation waves can be achieved as calibrated to this invention. The inventions sensing magneto structure is further configured via the hardware and software to measure or detect an alpha dominated level or state of the measured brain, at separate readings for each hemisphere, with a further means of determining whether or not a bioactive agent or stimulant drug was consumed by the measured animal by the following:
1. Measuring additional body readable measurements with the sensing (or an additional sensing) magneto structure;
2. Measuring the speed/rate/flow, and context/subject matter (and transitions between separate thought processes, in both a functional neurological interpretation and a left/right brain processing/creativity analysis (or the verse, which is a more organizational or productive cognitive mindset (as determinable by said rate and context of read speech or imagined or processed speech, as decoded)));
3. Valuing or calculating the measured and interpreted values of the said:
a. Brainwave osscilating alpha measurements;
b. Rate and context of imagined speech; and
c. Additional body measurements.

The three stated, a, b, and c, are calculated or processed in comparison to each other to generate a calculation indicative of whether or not a stimulant bioactive drug is contributory to the awakened mind state.

Additionally, the induction magnetometers (additional) may deliver wireless and remote delivery of electronic medicating osscilations which may be configured to create neural oscillation induced emotions, moods, or sensations in the targeted brain by directionally calibrating the field of the induction osscilating magnetic field(s) to specifically and technically target, stimulate, or otherwise influenced a specific component or area of the target brain as aimed. An additional sensing magneto inductor may be included, along with GPS locating means, to both create a specific coordinate position determination of the brain and parts, along with a computerized image or imaging of the said brain. Both the image of the brain and the location values aid the hardware/software configuration/system in its feature of being able to calibrate one or multiple separate magneto induction probe-fields at specific and small targeted said areas or components of the brain. The resulting reaction of the magneto-probed brain is dependent on the characteristics of both the combination of areas/parts stimulated, and such waveform/magnetic field osscilation properties which are configurable in a variety of different properties/measurements/features including but not limited to hertz, field strength, gauss, tesla, volts, ohms, etc, as will be readily configurable in one skilled in the art of neuro-feedback and brainwave reading/imaging/measuring technologies. Generally, higher frequency probes induce more production of neuron activity to the probed portion, with lower frequencies providing the verse. Such stimulation of the brain can be utilized as a method of brainwave entrainment. It can be used to repair damaged areas or components, and as a form of electronic bio-medicine that can be used in a large variety of purposes, including creating different and custom moods and cognitive states of the probe targeted brain. As implied, calibrated probes may be included the verse in this system to measure areas or parts of the brain. The combination of both the said input and output forms may be parallely computer controlled by the system in a closed loop or feedback system, which may also be configured to said computer controlling in combination of the speech input/output feature of this invention. One example of a computer function may extent to manipulating speech input or output parameters, based on probe input/output parameters in a processing algorithm structure that is ultimately customizeable, configurable, or manipulateable by an operator in specific value functions via a computer interface.

Phycological Therapy:

As prescribed and described previously herein, the combination of both the speech input/output configuration of this system, as well as the input/output may be used in a therapeutic setting by a remote operator who utilizes the input (microphone or transducer for the input sound(to be outputted by the outputting magento)) in combination with a verse, sound generation/speaker(transducer) output hardware configuration synonomous with the said, verse, read or measured input magneto portion. This is essentially, telepathy accomplished by the elements and features of this invention. Said, is put to practical and applicable use by engaging in otherwise voluntary telepathic speech communication accomplished via the input and output speech magnetos of this orbital configuration. The psychological therapist operators can dedicate long hours specific to patient clients, at a convenience only achievable by the remote and distanced application of this invention. This is convenient in both the sense that the patient client can receive continued treatment, at long and continued hours, from a distanced location, without the inconvenience of both having to travel to and from the therapist, and having to spend any schedule time away from the said therapist/machine operating agent. Although require more monetary resourcing for compensation of the therapy machine agents who are lil<ely employed by a jurisctional entity privileged by exemptions of the nations applicable law as a limited liability corporation (as necessary for liability issues associated with the technical and controversial features and elements of the machine such as wireless signal interfering conditions that may be subject to regulatory warning requirements of the federal communications corporation of the united states of America (or equivalent government telecommunications entities in other foreign states and countries). Usually, warnings are placed by the agencies on advertisements, product labels, physical, digital, and marketing electronic media, etc, that involve or feature any association with the product. Warnings are usually provided in the risk or potential evocation of any risk of any type of electronically or electromagnetically or wirelessly caused medical condition that may result as a result of consumer product use. Electromagnetically or electromechanically caused medical condition warning regulations are generally provided by said agencies, and while this invention may be not be available in a retail or box application, like cellular devices, the corporation providing psychological and telepathic therapy using this technology will likely be required to include a warning on media such as electronic or print, or radio advertisements of the product, notifying potentially patient clients of any type of medical condition that may potentially result from the electromagnetic or electronic elements embodied by this technology.

Clients receiving psychological treatment via the application of either or both both the mood probing and/or telepathic speech communication aspects of this invention can receive the treatment regimen at unconventional but necessary hours of their active and resting schedule. It is possible for a specific treatment program to be continuously applied to the client patient 24 hours a day, 7 days a week which may be therapeutically necessary. Multiple operators and/or shifts of agents of the organization can be effectively established to modulate both psychologically stimulating or manipulating speech [communication elements of telepathy, in conjunction with parallely applied mood modulating magneto field probes to specified portions of the brain or parts of the brain. Many different significant mental states are achievable by the agents of the organization, as customly configured and communication regulary between the client and his electronic caregiver to achieve the most comforting, relaxing, and mood responses supporting ultimate well-being experienced by the client.

The process of psychological mental state modulation of the mental state of a client is provided as follows in combined applications regulated against each other;

Telepathic Speech Treatment Portion:
a. Imagined speech reading is taken via input magnetometer;
b. Conversational input is induced in reply to the sensed imagined speech reading;

Repeat a and b in a repetitive loop perpetually until;
c. A psychological or behavioral response is generated or produced by the client.

Then, repeat steps a and b in another continuation loop perpetually until c is achieved again. Then, keep repeating as follows occasionally at random or discretional operating intervals (consistent with the patients treatment plan) to;
d. Take an additional reading of speech indicative of an experienced mood or desire by the patient client; and/or in combination with a reading of an input probe portion or array combination of probe magneto portions to similar or combinationally (with the speech reading) to indicate, said, a mood or desire of the patient client.

For example, a subject may exhibit biometric responses that he wishes for the speech portion of the treatment plan to be reduced in frequency and/or audibile intensity

(decibles) when he is conducting activities such as relaxing. The mechanical operating agent of the corporate organization will then become caused to responsively modulate the processing, transmission, induction, and generation of computer process and technological input/output operations of the system and treatment plan to accommodate or compensate the subjects desires.

Similarly, mood measurements are taken. The plurality of complex features, elements, protocals, and parameters forming the basis of this system and psychological treatment programe are used to generate a response-combination of inputting and outputting variables to fairly compensate the subject at hand. For example, if a stimulus in the subjects schedule causes him to become irritated or frustrated, these are generally considered to be negative cognitive states (which generally, measures are taken to counter them).

Variable Mood Component Probing.

The probing and reading system specific to areas or portion of the remotely imaged client brain may be technically and specifically configured to generate variable mood conditions of the patient as necessary to treat mood or psychotic disorders associated with such. One example is bipolar disorder, which is considered to be manic depressive and features brain state fluctuations and variabilities as said. Manic depressive patterns can be treated by probing specific areas or parts of the patients brain to effectively stabilize the patients measured mood anomalies, without the need for invasive anti-psychotic medications.

Part 2: Method of Detecting Opposing Magneto Propulsions to National Boundaries:

Questions and concerns of national security arise with the ability to undectably maneuver exampled satellite-magneto applications through vast outer space by conflicting countries at times of war. Warfare concerns are subjectable if opposing nations cause magnetometer or magneto induction satellites to unlawfully and maliciously enter enemy national boundaries from outer space.

A system is conceived for the purpose of monitoring regions of orbital outer space to prevent such enemy activities abusing this technology. The further demonstrated conception is a system for locating, detecting, and preventing, and ceasing, enemy or unauthorized magneto-generations from orbital satellites to enter the nation of concerns territorial boundaries located on global planet earth.

The invention calls for at least two proportional large in comparison to the features and capabilities of the magneto-satellite systems they achieve to adjust. At least two magneto-satellites are called for, as a minimum requirement for the process of this invention. However, it is more effective and likely that a large number of them are used for obvious efficiency and speed purposes.

The magentos move around a designated portional area of earths orbital space. The generation of the sensing means of the magnetos are configured to be perpendicularly aimed at the direction of expectation of entry of said surveyed direction and location of enemy probe fields. This is implied, as entry expaction aimed towards the direction of the planet earth, in a specified area relative to a countries national territorial boundaries. Additional sensing magnetos can be aimed in the relative or parallel direction.

Steps;

1. Magnetos move and actively producing sensing fields in a directional orientation;

2. Each sensing magneto receives a reading indicative of a potential illegal magneto entry application;

3. Each magento-sensor is either relocated and/or recalibrated by a computer controlled means responsive to the detection of a potential enemy magneto entry probe;

4. New readings are generated by each magento-sensing-satellite;

5. Repeat 3 and 4 in a loop or repletion continuously and perpetually, in a functional processing system that regulates the said computer controlling (of calibration and movement) until finally;

6. Based on any and/or all of the prior steps in regards to readings, calculations, movements, calibrations, processes, functions, etc, of the system, a definite, specified, or estimated location of the directional orientation location parameters of a potential enemy magneto-satellite is calculated. Then;

7. At least one sensing magneto is caused to move along the length of that direction in space relative to the planet at a more or less constant or static calibration of reading, measuring the suspected generated field as it increases in detection values as the sensing magneto-satellite becomes closer and closer to the suspected location; in combination with the application of at least one second magneto to do the same in the perpendicular directional orientation of the said suspected location until;

8. Readings from both sensing magnetos determine the location of the enemy magneto-satellite.

Once the enemy magnetometer is located, it can be destroyed or analyzed.

It should be noted that the operations of this portion of the invention in regards to locating an enemy satellite are varied in operation (movement and calibration adjustments) to accommodate a moving enemy satellite.

1. Therapy involving a long ranged magnetometer including auditory speech hallucinations.

2. Therapy involving a long ranged magnetometer probing brain components to induce emotional or medical experiences.
